SEQUENCE LISTING (3 sequences)

SEQ ID NO: 1 - DNA, 7 bp, Artificial
  Peptide Nucleic Acid
tcctttt

SEQ ID NO: 2 - DNA, 7 bp, Artificial
  artificial DNA sequence
aggaaaa

SEQ ID NO: 3 - DNA, 11 bp, Artificial
  artificial DNA sequence
ttaggaaaata

(19) United States
(12) Patent Application Publication
(10) Pub. No.: US 2012/0282709 A1
(43) Pub. Date: Nov. 8, 2012

(54) METHOD AND DEVICE FOR DNA SEQUENCE ANALYSIS USING MULTIPLE PNA

(76) Inventors: Byung Chul Lee, Seoul (KR); Jin Sik Kim, Incheon (KR); Hyun Joon Shin, Seoul (KR); Sang Youp Lee, Seoul (KR); Ji Yoon Kang, Seoul (KR)

(21) Appl. No.: 13/496,059

(22) PCT Filed: Sep. 14, 2009
(86) PCT No.: PCT/KR2009/005210

§ 371 Date: Mar. 14, 2012

(51) Int. Cl. G01N 21/64; G01N 21/76; C07K 1/26; B82Y 5/00; B82Y 20/00

(52) U.S. Cl. 436/501; 422/69; 204/451; 977/774; 977/920

(57) ABSTRACT

Provided are a DNA sequence analysis method of high precision providing improved optical limits by detecting wavelengths of lights emitted from labels in the state where a DNA is electrically tethered and completely stretch, and a nanodevice chip for automating the method. Also provided are a DNA sequence analysis method capable of removing binding errors through complementarily binding between a plurality of peptide nucleic acids (PNAs) labeled with labels emitting lights of different wavelengths and a target DNA to be sequenced, and resolving the limit in optical spatial resolution.

### TECHNICAL FIELD

The present disclosure relates to a method and a device for DNA sequence analysis using multiple peptide nucleic acids (PNAs).

### BACKGROUND ART

Currently, the most commonly used DNA sequence analysis method is based on the Sanger method. The Sanger method is a technique whereby DNA strand elongation by polymerase is terminated using dideoxynucleotides (ddNTPs) and the resulting double-stranded DNA is separated by gel electrophoresis to analyze the base sequence corresponding to each dideoxynucleotide. The dideoxynucleotides have a 3′-hydrogen (H) group, not a 3′-hydroxyl (OH) group required for the formation of phosphodiester bond between two nucleotides, thus terminating DNA polymerization. Although existing sequence analysis methods based on the Sanger method provide reliable results, they require a lot of time and cost. In addition, they are inefficient for detection of a single-nucleotide polymorphism (SNP) because polymerase chain reaction (PCR) and Sanger sequencing have to be repeated several times.

Other sequence analysis methods include a method based on mass spectrometry instead of electrophoresis using biotin-ddNTPs rather than ddNTPs labeled with fluorescent dyes, a PCR direct sequencing method of using helicase, a pyrosequencing method of detecting pyrophosphate (PPi) released during DNA synthesis, a bulk-fluorescence DNA sequencing-by-synthesis method of using deoxynucleotides (dNTPs) labeled with fluorescent dyes to synthesize several DNA molecules, a single-molecule DNA sequencing of using dNTPs labeled with fluorescent dyes to synthesize a single DNA molecule, a sequencing by hybridization method of determining sequences by hybridizing randomly fragmented pieces of a DNA molecule and linking numerous repeating sequences using a computer, and a massively parallel sequencing with stepwise enzymatic ligation and cleavage method of determining sequences by attaching and detaching specific fragments to and from a DNA molecule. However, these methods are inapplicable to a long sequence since they are based on synthesis using NTPs labeled with fluorescent dyes.

Other methods for analysis of long sequences include a nanopore DNA sequencing method of forming very small nanopores in a lipid membrane provided between two aqueous solutions and passing DNA molecules therethrough, and a hybridization-assisted nanopore sequencing (HANS) method of hybridizing a DNA molecule with specific fragments of a DNA sequence and passing it through nanopores. However, these methods have lower detection limit than the fluorescence-based Sanger method since the signals are detected electrically.

Optical detection is known to provide the best sensitivity. With the development of the single photon detector, detection of a single molecule through measurement of fluorescence has become possible. However, the light signal emitted from the single molecule is very low in intensity and there is a limit in improving the detection efficiency owing to the noises from nearby light sources or occurring during signal processing. The above-described sequence analysis methods based on detection using fluorescent molecules have the problem that, since a large amount of fluorescent dye is added to the sample to be analyzed for polymerization with DNA, the unpolymerized fluorescent molecules result in noise signals. Although washing is performed to remove the noise signals, the noise signals cannot be removed completely since some fluorescent molecules are non-specifically bound to the sample surface.

Another problem is that error may occur when the fluorescent molecule is attached to the DNA. Since the fluorescent molecule is not attached to the base sequence to be analyzed 100%, detection error cannot be avoided. In addition, the use of a DNA structure such as dNTP labeled with a fluorescent molecule as a probe is problematic in that the DNA probe may be denatured or lose activity with time since it is very unstable biologically and chemically against, for example, nucleases.

As a method allowing for analysis of a long DNA with fast detection speed and high detection limit based on fluorescence, the optical DNA mapping technique has become an integral process. In the optical DNA mapping, it is important to stretch the coiled DNA since the limit of optical detection depends on the degree of coiling of the DNA. For this, two methods are studied presently. The first method is to tether a DNA stretched by the molecular combing technique and then bind fluorescent materials to desired base sequences for optical detection. Although this method allows for reading of multiple sequences at the same time using different fluorescent materials, the DNA can be stretched only up to 70% of its full length and automation is impossible since the DNA cannot be tethered at a desired position. The second method is to attach fluorescent materials to a DNA stretched using mechanical means and pass the stretched DNA through a microchannel so as to analyze base sequence by measuring the presence of the fluorescent material using a laser and an optical detector. This method is restricted in improving the detection limit since the DNA cannot be stretched 100% because one end of which is not tethered and the end portion of the DNA strand is undetectable since it is coiled.

### DISCLOSURE

### Technical Problem

The present disclosure is directed to providing a DNA sequence analysis method of high precision providing improved optical limits by detecting the wavelengths of lights emitted from labels in the state where a DNA is electrically tethered and completely stretch, and a nanodevice chip for automating the method.

The present disclosure is also directed to providing a DNA sequence analysis method capable of removing binding errors through complementarily binding between a plurality of peptide nucleic acids (PNAs) labeled with labels emitting lights of different wavelengths and a target DNA to be sequenced, and resolving the limit in optical spatial resolution.

### Technical Solution

In one general aspect, the present disclosure provides a nanodevice chip comprising: two units comprising two DNA sample reservoirs connected via a microchannel; and a plurality of nanochannels connecting the microchannel of each unit, wherein the cross section of the nanochannel is in the form of a trapezoid, the nanochannel has nanohorn structures formed intermittently along the nanochannel, and the nanohorn structure protrudes from both upper corners of the trapezoid.

In another general aspect, the present disclosure provides a DNA sequence analysis method using the nanodevice, comprising: loading a DNA sample to be sequenced in the DNA sample reservoir of one unit; moving the DNA sample through the microchannel of the unit by applying an electric field below 20 kV/m in a direction from the DNA sample reservoir to the other DNA sample reservoir of the unit; moving the DNA sample from the microchannel into the nanochannel by applying an electric field below 20 kV/m in a direction from the unit to the other unit parallel to the nanochannel; and applying an electric field higher than 20 kV/m in parallel to the nanochannel, so that the DNA is stretched, with one end of the DNA being tethered by the nanohorn structure in the nanochannel while the other end moves in the nanochannel.

In another general aspect, the present disclosure provides a DNA sequence analysis method, comprising: complementarily binding a plurality of peptide nucleic acids (PNAs) labeled with labels emitting lights of different wavelengths to a target DNA to be sequenced; moving the DNA into a nanochannel having a nanohorn structure; applying an electric field higher than 20 kV/m to the nanochannel, so that the DNA is stretched, with one end of the DNA being tethered by the nanohorn structure in the nanochannel while the other end moves in the nanochannel; and detecting the wavelengths of the lights emitted from the labels of the plurality of PNAs complementarily bound to the DNA.

### Advantageous Effects

The following effects may be obtained by using the nanodevice chip and the DNA sequence analysis method of the present disclosure.

First, it is possible to detect optical signals with high spatial resolution in real time, thereby achieving detection with high sensitivity, high efficiency and low noise.

Second, by integrating nanochannels which may temporarily tether a DNA and stretch it, it is possible to allow for control of detection speed and reduction of PCR cost.

Third, it is possible to detect and remove peptide nucleic acid (PNA) binding errors using a plurality of PNAs and fluorescent labels as well as the fluorescence resonance energy transfer (FRET) method, and to avoid the use of exquisite and expensive optical filters by increasing wavelength shift.

In addition, the sequence analysis is automated using the nanodevice chip in which a plurality of nanochannels are integrated as well as a multi-channel laser and an optical system, thereby contributing to personal genome mapping, personalized medicine and treatment.

### DESCRIPTION OF DRAWINGS

FIG. 1 schematically shows a nanodevice chip according to an embodiment of the present disclosure.

FIG. 2 schematically shows (a) movement of a DNA sample from a reservoir to a nanochannel inlet through a microchannel, (b) movement of the DNA sample from the nanochannel inlet to a nanohorn structure in a nanochannel by an electric field applied in parallel to the nanochannel, and (c) tethering and stretching of a DNA in the nanochannel by an electric field applied to the DNA sample, according to an embodiment of the present disclosure.

FIG. 3 schematically shows a nanodevice chip fabrication process according to an embodiment of the present disclosure.

FIG. 4 shows the cross section of a nanochannel prepared according to an embodiment of the present disclosure.

FIG. 5 schematically illustrates sequencing of a target DNA to be sequenced which is complementarily bound to a plurality of peptide nucleic acids (PNAs) labeled with labels emitting lights of different wavelengths according to an embodiment of the present disclosure. (a) shows the case where the distance between the target base sequences is longer than the optical resolution, and (b) shows the case where the distance between the target base sequences is smaller than the optical resolution.

FIG. 6 schematically shows polymerization of a single-stranded PNA labeled with a fluorescent label with a target DNA according to an embodiment of the present disclosure.

FIG. 7 schematically shows polymerization of a double-stranded PNA obtained by linking PNAs having the same base sequence using a linker with a target DNA according to an embodiment of the present disclosure. (a) shows the case where only one end of the strand is labeled, and (b) shows the case where both ends of the strand are labeled with the same label.

FIG. 8 schematically illustrates base sequencing by attaching a fluorescence resonance energy transfer (FRET) donor fluorescent label and a FRET acceptor fluorescent label to a double-stranded PNA linked by a linker according to an embodiment of the present disclosure. (a) shows the case where the PNA sequence complementarily binds to the base sequence of the target DNA perfectly, and (b) shows the case where the PNA sequence binds to the base sequence of the target DNA imperfectly.

FIG. 9 schematically illustrates a process of loading a DNA sample on a nanodevice chip and applying an electric field according to an embodiment of the present disclosure.

FIG. 10 shows the degree of stretching of a DNA depending on the intensity of an applied electric field according to an embodiment of the present disclosure.

### BEST MODE

The present disclosure provides a nanodevice chip comprising: two units comprising two DNA sample reservoirs connected via a microchannel; and a plurality of nanochannels connecting the microchannel of each unit, wherein the cross section of the nanochannel is in the form of a trapezoid, the nanochannel has nanohorn structures formed intermittently along the nanochannel, and the nanohorn structure protrudes from both upper corners of the trapezoid.

As used herein, the microchannel refers to a channel with cross-sectional transverse and longitudinal lengths or a diameter smaller than 1 mm, and the nanochannel refers to a channel with transverse and longitudinal lengths smaller than 1 μm.

The present disclosure also provides a DNA sequence analysis method using the nanodevice chip. Specifically, the method comprises: loading a DNA sample to be sequenced in the DNA sample reservoir of one unit; moving the DNA sample through the microchannel of the unit by applying an electric field below 20 kV/m in a direction from the DNA sample reservoir to the other DNA sample reservoir of the unit; moving the DNA sample from the microchannel into the nanochannel by applying an electric field below 20 kV/m in a direction from the unit to the other unit parallel to the nanochannel; and applying an electric field higher than 20 kV/m in parallel to the nanochannel, so that the DNA is stretched, with one end of the DNA being tethered by the nanohorn structure in the nanochannel while the other end moves in the nanochannel.

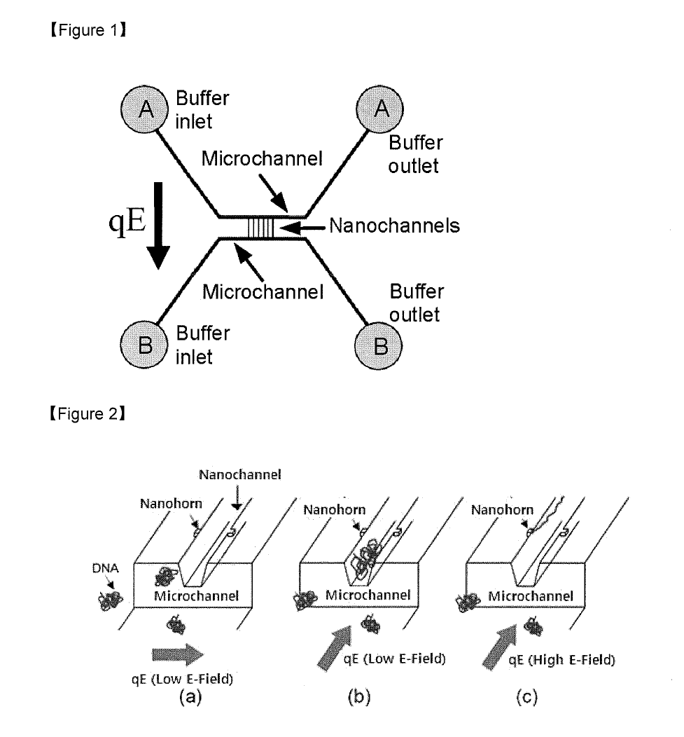

FIG. 1 schematically shows a nanodevice chip according to an embodiment of the present disclosure. The nanodevice chip comprises three portions: a DNA sample reservoir loading a DNA sample to be sequenced to which labeled peptide nucleic acids (PNAs) are complementarily bound; a microchannel serving as a passage for moving the DNA sample from the reservoir to a nanochannel inlet; and a nanochannel electrically tethering and completely stretching the DNA sample to allow for detection of wavelengths of lights emitted from the labels of the PNAs complementarily bound to the DNA. When the reservoir with a very large volume is directly connected to the nanochannel, the possibility of a buffer including the DNA sample entering to the nanochannel are very low. Thus, by connecting the reservoir to the nanochannel using the microchannel, so that the microchannel may serves as a passage for moving the DNA sample from the reservoir to the nanochannel inlet, the probability of the DNA sample from the DNA sample reservoir entering the nanochannel can be increased.

Since a DNA has a negatively charged phosphate group, it may move under the influence of an electric field. Thus, after the DNA sample to be sequenced is loaded in one DNA sample reservoir of one unit, the DNA sample may be moved from the reservoir through the microchannel of the unit by applying an electric field below 20 kV/m in a direction from the DNA sample reservoir to the other DNA sample reservoir of the unit. When the DNA sample is moved to the nanochannel inlet through the microchannel, the DNA sample may be moved from the microchannel into the nanochannel by applying an electric field below 20 kV/m in a direction from the unit to the other unit parallel to the nanochannel.

A long DNA exists in a supercoiled state in nature. For example, although 48.5-kbp λ-DNA has is 16.5 μm long when fully stretched, it normally exists in a wound state of 0.8-1 μm length. When the coiled DNA is moved into the nanochannel, it is stretched to some extent owing to the spatial confinement effect. However, the degree of DNA stretching in the nanochannel by the spatial confinement effect, determined by the width and height of the nanochannel, is up to about 80%. For example, when a nanochannel of a dimension of about 400 nm×400 nm is used, the DNA is stretched up to about 20%.

After the DNA is moved until one end of the DNA is located at the nanohorn structure by applying an electric field below 20 kV/m, when an electric field higher than 20 kV/m is applied in parallel to the nanochannel, the electric field is locally concentrated due to the nanohorn structure and dielectrophoresis (DEP) force is generated, as a result of which the one end of the DNA is temporarily tethered to the nanohorn structure. At the same time, electrostatic force exerted by the electric field makes the other, negatively charged, end of the DNA to move in the nanochannel. As a result, the DNA is stretched. When the DNA is stretched according to the method of the present disclosure, the DNA sequence can be accurately analyzed since the labels of the PNAs complementarily bound to the DNA do not overlap with each other and optical resolution is maximized.

FIG. 2 schematically shows (a) movement of the DNA sample from the reservoir to the nanochannel inlet through the microchannel, (b) movement of the DNA sample from the nanochannel inlet to the nanohorn structure in the nanochannel by the electric field applied in parallel to the nanochannel, and (c) tethering and stretching of the DNA in the nanochannel by applying the high electric field to the DNA sample, according to an embodiment of the present disclosure.

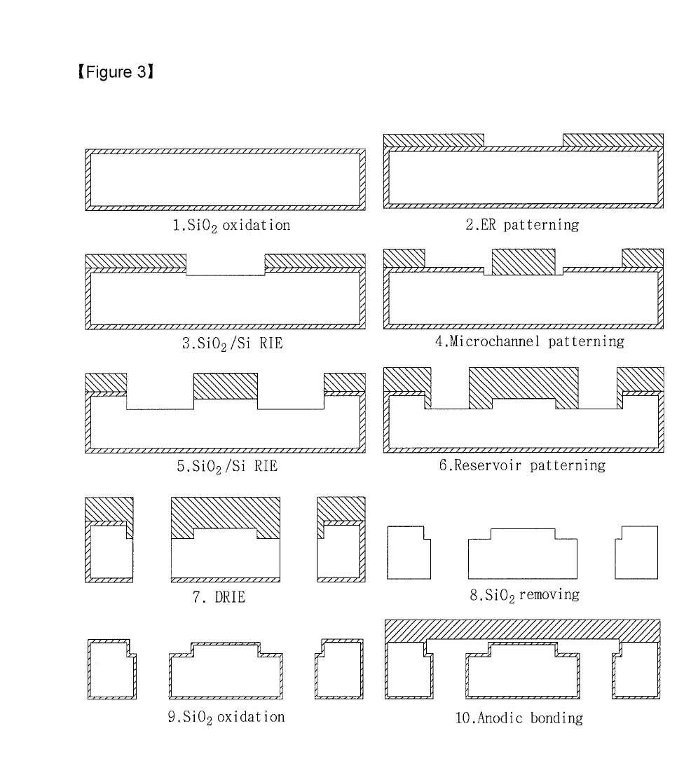

In an embodiment of the present disclosure, the nanodevice chip may be fabricated on a silicon substrate via a known silicon process and an anodic bonding method. Anodic bonding is a technique of fixing a conductor or a semiconductor on a glass substrate using strong electrostatic force resulting from the ion conductivity of the substrate. FIG. 3 schematically shows a nanodevice chip fabrication process according to an embodiment of the present disclosure.

First, forty parallel nanochannels are patterned on a silicon wafer by standard electron beam lithography and reactive-ion etching (RIE) is carried out. After the electron beam resist is removed, two microchannels are formed by standard photolithography or RIE. Then, four reservoirs are prepared by photolithography or deep reactive-ion etching (DRIE). Next, anodic bonding is carried out using a glass wafer to obtain the nanodevice chip according to an embodiment of the present disclosure. The nanochannel may comprise SiO₂.

The cross section of the nanochannel may be in the form of a trapezoid, with a top side of 100-500 nm, a bottom side of 100-500 nm and a height of 100-500 nm, more specifically with a top side of 450 nm, a bottom side of 200 nm and a height of 400 nm, but without being limited thereto. The nanohorn may have a depth of 5-30 nm, but without being limited thereto. When the cross-sectional area of the nanochannel is too small as compared to the nanohorn, the change of the electric field by the nanohorn may be only slight. The nanochannels may be provided with intervals from 500 nm to infinity. An infinite interval means that only one nanochannel is provided. When the distance between the nanochannels is smaller than 500 nm, problems may occur during bonding.

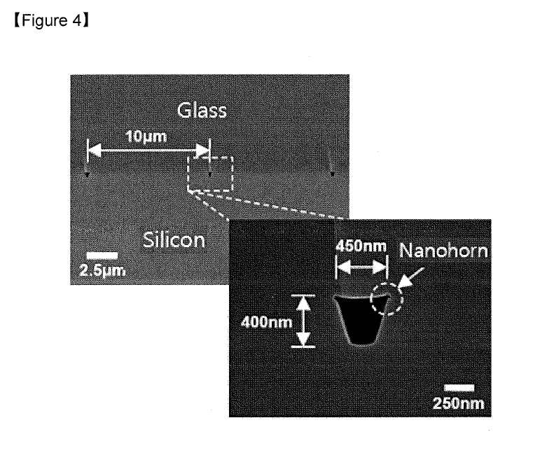

In an embodiment of the present disclosure, the nanohorn structures are formed intermittently along the nanochannel, and the nanohorn structure protrudes from both upper corners of the trapezoid when viewed from the cross section of the nanochannel. The nanohorn structure may be formed under high bonding temperature and pressure during the anodic bonding. Since the bonding temperature is 400° C., close to the glass transition temperature of 560° C., the top heat-resistant glass (Pyrex) may sag about 30 nm under the bonding pressure (1 kgf/cm²). FIG. 4 shows the cross section of the nanochannel prepared according to an embodiment of the present disclosure.

When an electric field higher than 20 kV/m is applied in parallel to the nanochannel, the electric field is locally concentrated due to the nanohorn structure and DEP force is generated, as a result of which the one end of the DNA is temporarily tethered to the nanohorn structure. At the same time, electrostatic attraction exerted by the electric field makes the other, negatively charged, end of the DNA to move in the nanochannel. As a result, the DNA is stretched.

When the electric field is removed, the DNA may be coiled again. Because of the spatial confinement effect by the nanochannel, the time required for the stretched DNA to be coiled again after the electric field is removed (relaxation time) is longer than that in a microchannel or in a free space. In an exemplary measurement, it took 10 seconds for the DNA to be coiled again after an electric field of 20 kV/m has been applied and then removed.

The DEP force confines a charged molecule within a space in “negative dielectrophoresis” state. Under low electric field, the DNA can move since the DEP force is lower than the electrophoretic force. But, when the electric field exceeds the critical electric field, the DNA is tethered to the nanohorn.

The present disclosure also provides a DNA sequence analysis method, comprising: complementarily binding a plurality of PNAs labeled with labels emitting lights of different wavelengths to a target DNA to be sequenced; moving the DNA into a nanochannel having a nanohorn structure; applying an electric field higher than 20 kV/m to the nanochannel, so that the DNA is stretched, with one end of the DNA being tethered by the nanohorn structure in the nanochannel while the other end moves in the nanochannel; and detecting the wavelengths of the lights emitted from the labels of the plurality of PNAs complementarily bound to the DNA.

The DNA sequence analysis method according to the present disclosure may be used to detect difference in DNA sequence between individuals for a DNA whose full-length information is known.

The PNA, having peptide bonds instead of the phosphodiester bonds of a DNA, may be specifically hybridized or polymerized with a DNA since it has adenine, thymine, guanine and cytosine residues like a DNA. A probe consisting only of polymerized DNAs has the problems of very low biological and chemical stability as well as denaturation and decreased reactivity of DNA with time. In contrast, the PNA having peptide bonds instead of phosphodiester bonds is highly stable. Also, since the peptide backbone is electrically neutral, stronger binding is possible during polymerization since the electrostatic repulsion is removed. For this reason, faster polymerization is possible, signal-to-noise (S/N) ratio is improved owing to high specificity, and biological and chemical stability is improved. The PNA may be bound to a double helical DNA in two ways. First, it may be inserted into the double strand structure of the DNA via Watson-Crick hydrogen bonds. Alternatively, it may be attached to the DNA, specifically beside the double strand structure of the DNA, via Hoogsteen hydrogen bonds.

In an embodiment of the present disclosure, the plurality of PNAs are labeled with labels emitting lights of two wavelengths. One of the labels may be labeled at the PNA complementarily bound to one or more target base sequences to be analyzed, and the other label may be labeled at the PNAs complementarily bound to the base sequences before or after the target base sequence. FIG. 5 schematically illustrates sequencing of a stretched DNA which is complementarily bound to a plurality of PNAs. In an exemplary embodiment, the PNA complementarily bound to the target base sequence of the DNA is labeled with a red fluorescent label (depicted as void circles in FIG. 5), and the PNAs complementarily bound to the base sequences before or after the target base sequence are labeled with a blue fluorescent label (depicted as solid circles in FIG. 5) for analysis of binding errors of the PNA labeled with the red fluorescent label.

In an embodiment of the present disclosure, when the distance between the target base sequences is longer than the optical resolution (about 400 nm) as in (a), three PNAs labeled with fluorescent labels emitting (red and blue) lights of two different wavelengths are labeled as a set (That is, the PNA complementarily bound to the target base sequence is labeled with the red fluorescent label, and the PNAs complementarily bound to the sequences before and after the target base sequence are labeled with the blue fluorescent label.) per each target base sequence, so as to allow detection of the distances between the target base sequences using a double laser channel or two laser channels.

In an embodiment of the present disclosure, when the distance between the target base sequences is shorter than the optical resolution as in FIG. 5 (b), a plurality of PNAs may be labeled with labels emitting lights of four wavelengths, a first label among the labels being labeled at the PNA complementarily bound to a first target base sequence, a second label being labeled at the PNA complementarily bound to a second target base sequence, a third label being labeled at the PNA complementarily bound to a third base sequence distant within the optical resolution from the first target base sequence and distant beyond the optical resolution from the second target base sequence, and a fourth label being labeled at the PNA complementarily bound to before and after the first to third base sequences.

When the target base sequences at locations A and B are distant within the optical resolution, if a yellow fluorescent label is labeled at the location A and a red fluorescent label is labeled at the location B, the two fluorescent lights are superposed since the distance between the locations is smaller than the optical resolution. In this case, when a location C distant from the location A by the minimum optical resolution is label with a green fluorescent label, the fluorescent lights from the location A and the location C are not superposed whereas those from the location B and the location C are superposed since the distance between the locations is smaller than the optical resolution. Accordingly, it can be identified how far the location A and the location B are distant from the location C.

Specifically, a DNA sequence analysis method may comprise: (a) complementarily binding PNAs labeled with a yellow fluorescent label (depicted as horizontally striped circle in FIG. 5) and a red fluorescent label (depicted as void circle in FIG. 5), respectively, to target base sequences at location A and location B; (b) detecting binding errors of the PNAs bound at the locations A and B by complementarily binding PNAs labeled with a blue fluorescent label (depicted as solid circle in FIG. 5) before and after the locations A and B, respectively; (c) complementarily binding a PNA labeled with a green fluorescent label (depicted as vertically striped circle in FIG. 5) at location C which is distant within the optical resolution from the location B and by the minimum optical resolution from the location A; and (d) detecting binding errors of the PNA bound at the location C by complementarily binding PNAs labeled with a blue fluorescent label (depicted as solid circle in FIG. 5) before and after the location C. Since the fluorescent labels emit lights of four wavelengths, a quadruple laser channel may be used for the detection.

In an embodiment of the present disclosure, the label may be a fluorescent label, a luminescent label, a chemiluminescent label, a fluorescence resonance energy transfer (FRET) label, a quantum dot label or a metal label. The fluorescent label may be an organic fluorescent label such as Cy-5, Cy-3, Alexa 647, Alexa 488, TOTO or the like, a biotin-conjugated label, tetramethylrhodamine (TMR), tetramethylrhodamine isothiocyanate (TMRITC), x-rhodamine, Texas Red, or the like.

In an embodiment of the present disclosure, the PNA may comprise 4 or more base sequences, specifically 4-9 base sequences, although not being limited thereto. When the PNA comprises less than 4 base sequences, the possibility of non-specific binding increases. Also, when the PNA comprises more than 9 base sequences, synthesis of the PNA becomes very difficult and cost is increased.

In an embodiment of the present disclosure, the PNA may be single-stranded or double-stranded with two PNAs having the same base sequence being linked by a linker. When the PNA is double-stranded, binding errors may be further reduced.

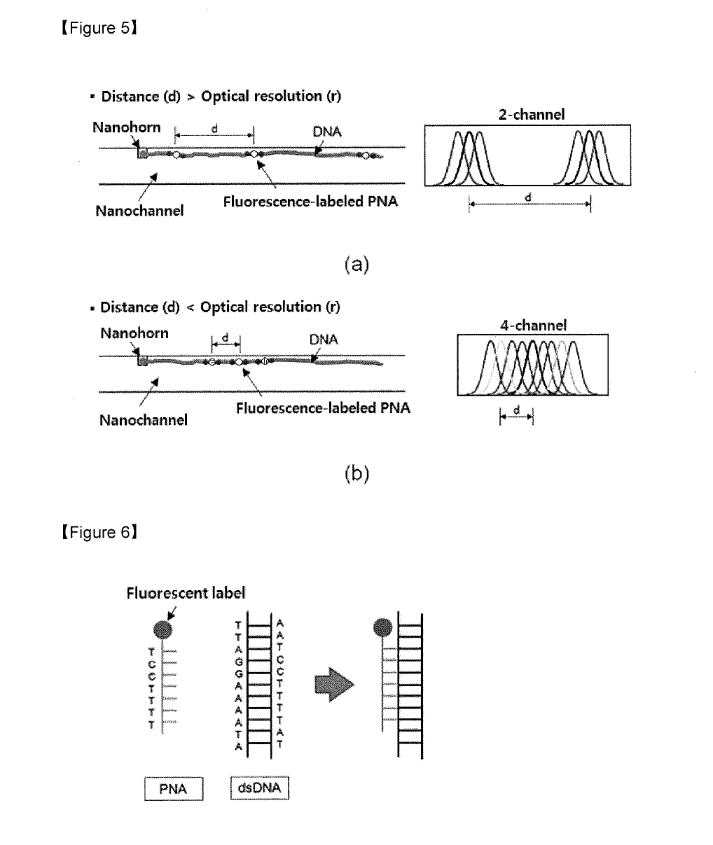

In an exemplary embodiment of the present disclosure, when the PNA is single-stranded, it may comprise 7 base sequences (TCCTTTT) as shown in FIG. 6 and may be bound to a double-stranded DNA of 7 base sequences (AGGAAAA) via Hoogsteen hydrogen bonds after a fluorescent label is labeled at the end portion.

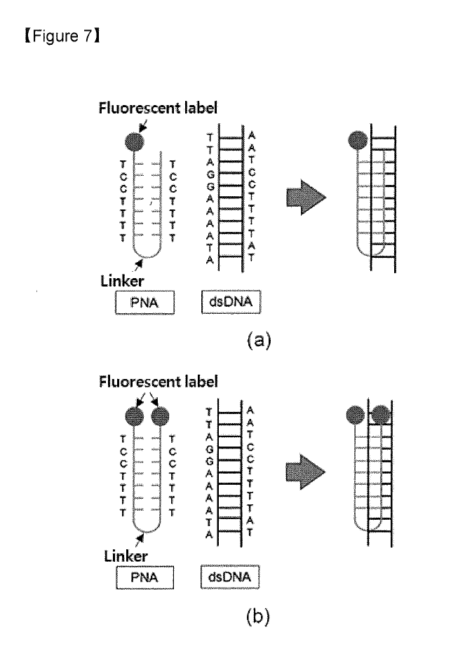

In another embodiment of the present disclosure, when the PNA is double-stranded with two PNAs having the same base sequence being linked by a linker, it may be bound to a double-stranded target DNA via both Hoogsteen hydrogen bonds and Watson-Crick hydrogen bonds to reduce binding errors. Only one of the strands may be labeled, or both strands may be labeled with the same labels. For example, as shown in FIG. 7 (a), a PNA comprising 7 base sequences (TCCTTTT) and having a fluorescent label attached at the end portion may be linked with the same PNA comprising 7 base sequences and then bound to a target DNA. Also, as shown in FIG. 7 (b), the fluorescent labels may be attached at the end portion of both PNA strands to double the light emission.

In another embodiment of the present disclosure, when the PNA is double-stranded with two PNAs having the same base sequence being linked by a linker, the end portion of one strand may be labeled with a fluorescence resonance energy transfer (FRET) donor label and the portion of the other strand may be labeled with a FRET acceptor label. FRET is a phenomenon in which a fluorescent donor excited by absorbing light energy emits fluorescence while nonradiative energy is transferred to a nearby acceptor within several nanometers through resonance and the acceptor emits long-wavelength fluorescence. The long-wavelength fluorescence can be detected when the distance between the donor and the acceptor is within several nanometers.

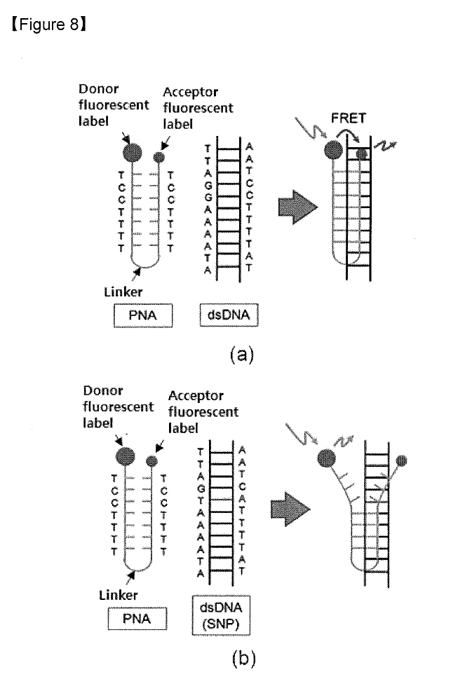

For example, as shown in FIG. 8 (a), a PNA comprising 7 base sequences (TCCTTTT) and having a donor fluorescent label attached at the end portion may be linked with the same PNA comprising 7 base sequences by a linker and then polymerized with a target DNA. Then, when the donor is excited by radiating short-wavelength light, long-wavelength fluorescence is detected as a result of energy transfer from the donor to an acceptor if the PNA sequence is complementary bound to the target DNA base sequence and the distance from the donor to the acceptor is within several nanometers. If there exists single-nucleotide polymorphism (SNP) in the target DNA as shown in FIG. 8 (b) and the 7 base sequences (TCCTTTT) of the PNA fails to complementarily bind to the target DNA base sequence, the FRET phenomenon does not occur because the donor fluorescent label is distant from the acceptor fluorescent label and the long-wavelength fluorescence is not detected. In this manner, binding errors or base sequence variation can be analyzed.

In an embodiment of the present disclosure, the use of exquisite and expensive optical filters can be avoided since the wavelength shift is increased when FRET is employed.

### MODE FOR INVENTION

The movement of DNA was confirmed through experiments. FIG. 9 schematically illustrates a process of loading a DNA sample on a nanodevice chip and applying an electric field according to an embodiment of the present disclosure.

First, standard TBE buffer was filled in the channels of the nanodevice chip. 1×TBE solution containing 4% (v/v) β-mercaptoethanol and 0.2% (w/v) POP6 was used as the buffer in order to suppress electroosmotic flow. The viscosity of the buffer was measured as 1.02 cP at room temperature (24° C.), and the conductivity of the buffer was measured as 64.2 μS/cm from an impedance analyzer. The buffer was degassed for about 1 hour using an ultrasonicator and a vacuum-pumped desiccator. The standard buffer was loaded into the reservoirs 1, 2 and then into the reservoirs 3, 4 at the opposite side. After filling the standard buffer, the DNA sample was loaded into the reservoir 1. An electric field was applied in a direction from the reservoir 1 to the reservoir 3 so that the DNA sample could move through the microchannel. Then, an electric field was applied in parallel to the nanochannel so that the DNA sample could move from the microchannel to the nanochannel.

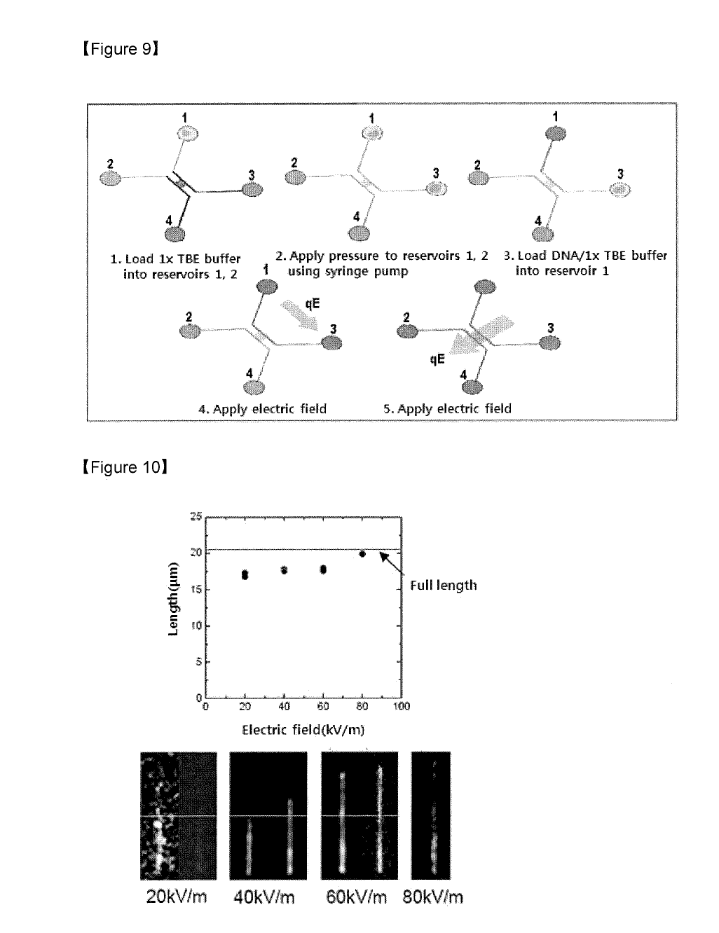

Electric field from 0.4 to 80 kV/m was used. Under low electric field below 20 kV/m, the average mobility of λ-DNA was 4.51×10⁻⁹ m²/Vsec. When the intensity of the electric field was increased above 20 kV/m, the DNA molecule moved faster along the nanochannel. The DNA molecule was tethered in the middle of the nanochannel by the nanohorn structure and was simultaneously stretched along the nanochannel. The length of the supercoiled DNA in the microchannel was 1.04 μm. The length of the DNA molecule elongated in the nanochannel due to the spatial confinement effect was 3.90 μm (20% of full length), and the length of the DNA tethered by dielectrophoresis (DEP) force owing the nanohorn structure and stretched by electrostatic force under an electric field of 60 kV/m was 17.94 μm (92% of full length). The DNA molecule could be stretched about 100% by increasing the intensity of the electric field. FIG. 10 shows the degree of stretching of the DNA molecule depending on the intensity of the applied electric field.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A nanodevice chip comprising:
two units comprising two DNA sample reservoirs connected via a microchannel; and
a plurality of nanochannels connecting the microchannel of each unit,
wherein the cross section of the nanochannel is in the form of a trapezoid,
the nanochannel has nanohorn structures formed intermittently along the nanochannel, and
the nanohorn structure protrudes from both upper corners of the trapezoid.

2. The nanodevice chip according to claim 1, wherein the nanochannel comprises SiO₂.

3. A DNA sequence analysis method using the nanodevice chip according to claim 1, comprising:
loading a DNA sample to be sequenced in the DNA sample reservoir of one unit;
moving the DNA sample through the microchannel of the unit by applying an electric field below 20 kV/m in a direction from the DNA sample reservoir to the other DNA sample reservoir of the unit;
moving the DNA sample from the microchannel into the nanochannel by applying an electric field below 20 kV/m in a direction from the unit to the other unit parallel to the nanochannel; and
applying an electric field higher than 20 kV/m in parallel to the nanochannel, so that the DNA is stretched, with one end of the DNA being tethered by the nanohorn structure in the nanochannel while the other end moves in the nanochannel.

4. A DNA sequence analysis method, comprising:
complementarily binding a plurality of peptide nucleic acids (PNAs) labeled with labels emitting lights of different wavelengths to a target DNA to be sequenced;
moving the DNA into a nanochannel having a nanohorn structure;
applying an electric field higher than 20 kV/m to the nanochannel, so that the DNA is stretched, with one end of the DNA being tethered by the nanohorn structure in the nanochannel while the other end moves in the nanochannel; and
detecting the wavelengths of the lights emitted from the labels of the plurality of PNAs complementarily bound to the DNA.

5. The DNA sequence analysis method according to claim 4,
wherein the cross section of the nanochannel is in the form of a trapezoid, the nanohorn structure is formed intermittently along the nanochannel, and the nanohorn structure protrudes from both upper corners of the trapezoid.

6. The DNA sequence analysis method according to claim 4,
wherein the plurality of PNAs are labeled with labels emitting lights of two wavelengths, one of the labels being labeled at the PNA complementarily bound to one or more target base sequences to be analyzed and the other label being labeled at the PNAs complementarily bound to the base sequences before or after the target base sequence.

7. The DNA sequence analysis method according to claim 4,
wherein, when the distance between the target base sequences is shorter than the optical resolution, the plurality of PNAs are labeled with labels emitting lights of four wavelengths, a first label among the labels being labeled at the PNA complementarily bound to a first target base sequence, a second label being labeled at the PNA complementarily bound to a second target base sequence, a third label being labeled at the PNA complementarily bound to a third base sequence distant within the optical resolution from the first target base sequence and distant beyond the optical resolution from the second target base sequence, and a fourth label being labeled at the PNA complementarily bound to before and after the first to third base sequences.

8. The DNA sequence analysis method according to claim 4, wherein the label is a fluorescent label, a luminescent label, a chemiluminescent label, a fluorescence resonance energy transfer (FRET) label, a quantum dot label or a metal label.

9. The DNA sequence analysis method according to claim 4, wherein the PNA comprises 4-9 base sequences.

10. The DNA sequence analysis method according to any claim 4,
wherein the PNA is single-stranded or double-stranded with two PNAs having the same base sequence being linked by a linker.

11. The DNA sequence analysis method according to claim 10, wherein in the double-stranded PNA linked by the linker, the end portion of only one strand is labeled with a label or the end portions of both strands are labeled with the same label.

12. The DNA sequence analysis method according to claim 10, wherein in the double-stranded PNA linked by the linker, the end portion of one strand is labeled with a fluorescence resonance energy transfer (FRET) donor label and the portion of the other strand is labeled with a FRET acceptor label.

13. The DNA sequence analysis method according to claim 4, wherein the wavelengths of the lights emitted from the labels of the plurality of PNAs are detected using a multiple laser channel.
